(19) United States
(12) Patent Application Publication
(10) Pub. No.: US 2007/0098666 A1
(43) Pub. Date: May 3, 2007

(54) Citric acid ester

(75) Inventors: Ansgar Behler, Bottrop (DE); Werner Seipel, Hilden (DE); Norbert Boyxen, Kempen (DE); Daniela Prinz, Dormagen (DE)

Correspondence Address: COGNIS CORPORATION;PATENT DEPARTMENT, 300 BROOKSIDE AVENUE, AMBLER, PA, 19002, US

(73) Assignee: COGNIS IP MANAGEMENT GMBH, DUESSELDORF (DE)

(21) Appl. No.: 10/554,968

(22) PCT Filed: Apr. 21, 2004
(86) PCT No.: PCT/EP04/04209

§ 371 Date: Dec. 15, 2006

(30) Foreign Application Priority Data
Apr. 30, 2003 (DE) 103 19 401.0

(51) Int. Cl. A61K 8/37

(52) U.S. Cl. 424/70.31

(57) ABSTRACT

A mixture of citric acid esters of ethoxylated alcohols wherein the residue of the alcohol contains from 12 to 18 carbon atoms ethoxylated with from 5 to 9 ethoxy groups and a ratio by weight of monoester to diester in the mixture of citric acid esters in a range of 3:1 to 10:1. The mixture contains less than 10% by weight of unesterified citric acid. The citric acid ester mixture provides improved foaming ability and reduced lachrimatory properties when compared to citric acid esters containing a different ratio of monoester to diester and containing longer or shorter chain length alcohol residues.

### FIELD OF THE INVENTION

This invention relates to selected citric acid ester mixtures of selected ethoxylated alcohols with a special ratio of monoester to diester, to a process for their production and to their use—optionally in admixture with other surfactants—for the production of high-foaming cosmetic preparations with a low irritation potential.

### PRIOR ART

Citric acid esters—also known as alkylether citrates—are well-known compounds which have already been used in cosmetic products. For example, European patent application EP 282 289 A1 describes cosmetic compositions which contain monoalkyl citric acid salts of C₁₀₋₁₈ alcohols ethoxylated with 1 to 7 mol EO. According to this document, particularly high monoester contents of >95% of the citric acid acids are desirable and can be obtained by reaction of citric anhydride with the corresponding ethoxylated alcohols.

International patent application WO 94/10970 describes a solubilizer containing monoalkyl citrates with C₇₋₁₀ alkyl groups as an ingredient of perfumes and cosmetic compositions, such as fabric and body care and cleaning preparations. Published European patent application EP 199 131 A describes citric acid esters of C₈₋₂₀ alcohols ethoxylated with 1 to 20 mol EO. The esters may be mono-, di- or triesters. According to this document, citric acid esters produced from 1 mol citric acid and 2 mol of an alcohol mixture of C₁₁, C₁₂ and C₁₃ alcohols ethoxylated with 7 mol EO show low irritation potential and acceptable foaming behavior.

The use of citric acid esters for improving the removability of oil-containing cosmetic compositions by washing is known from European patent EP 852 944 B1. According to this document, the citric acid esters are esters of C₁₂₋₁₈ alcohols ethoxylated with 5 to 30 mol EO; the esters may be mono-, di and/or triesters. According to the Examples, the mono- or diesters of coconut oil alcohol—which always contains unsaturated alcohols also—ethoxylated with 7 or 9 mol EO are particularly suitable.

According to U.S. Pat. No. 6,413,527, nanoemulsions containing citric acid esters of C₈₋₂₂ alcohols ethoxylated with 3 to 9 mol EO have good hair- and skin-moisturizing properties, mono-, di- and/or triesters being equally suitable.

Finally, according to the article by R. Diez et al. in: Proceedings, 4^{th} World Surfactant Congress, Barcelona (1996), Vol. 2, pp. 129 et seq, alkylether citrates are anionic surfactants which are suitable for cosmetic applications. Citric acid esters of lauryl alcohol with various degrees of ethoxylation (3, 6 and 9), which may be present as mono-, di and/or triesters, were investigated. The monoesters cited in this article are a mixture of mono- and diesters in a ratio of 5:1. The esters show, for example, moderate foaming behavior, the monoesters of lauryl alcohol ethoxylated with 3 and 6 mol ethylene oxide showing better foaming behavior than the diesters whereas the esters with 9 mol ethylene oxide are better as diesters than the monoesters.

However, the products known from the prior art are attended by various disadvantages. Thus, the citric acid esters known from the prior art—particularly in combination with other surfactants—often lack the foam behavior consumers want from cosmetic preparations, particularly shampoos and bath additives.

Accordingly, the problem addressed by the present invention was to provide citric acid esters which would show very good foam behavior both in regard to foaming kinetics and in regard to foam behavior after prolonged time periods. In addition, the citric acid esters would have hardly any irritation potential. The invention also sought to provide citric acid esters which would lend themselves to clear formulation with other surfactants typically encountered in cosmetic products. Finally, the citric acid esters according to the invention would have high surface activity of their own.

### DESCRIPTION OF THE INVENTION

The present invention relates to citric acid ester mixtures of ethoxylated alcohols corresponding to general formula (I):
R¹O(CH₂CH₂O)ₙH (I)
in which R¹ is an alkyl group and n is the degree of ethoxylation, characterized in that R¹ is a linear alkyl group derived from a fatty alcohol mixture containing 45 to 75% by weight C₁₂, 15 to 35% by weight C₁₄, 0 to 15% by weight C₁₆ and 0 to 20% by weight C₁₈ alcohol and n is a number of 5 to 9, with the proviso that the ratio by weight of monoester to diester in the citric acid ester mixtures is in the range from 3:1 to 10:1.

The present invention also relates to a process for the production of the citric acid ester mixtures of ethoxylated alcohols according to the invention corresponding to general formula (I):
R¹O(CH₂CH₂O)ₙH (I)
in which R¹ is a linear alkyl group derived from a fatty alcohol mixture containing 45 to 75% by weight C₁₂, 15 to 35% by weight C₁₄, 0 to 15% by weight C₁₆ and 0 to 20% by weight C₁₈ alcohol and n is a number of 5 to 9, with the proviso that the ratio by weight of monoester to diester in the citric acid ester mixtures is in the range from 3:1 to 10:1, characterized in that the citric acid is esterified with the alcohol ethoxylates of formula (I) in a molar ratio of 0.9:1 to 1.1:1 and more particularly 1:1.

The present invention also relates to the use of citric acid ester mixtures of ethoxylated alcohols corresponding to general formula (I), optionally in admixture with other surfactants, for the production of foaming, skin-friendly cosmetic preparations.

The citric acid ester mixtures selected in accordance with the invention surprisingly show both excellent foam behavior and no irritation potential with respect to the skin, even in combination with other surfactants. The better irritation potential compared with diesters of citric acid is particularly surprising because surfactants containing anionic groups (carboxylate group) show worse irritation potentials than surfactants containing nonionic groups (ester group).

Citric Acid Ester Mixtures

The citric acid ester mixtures according to the invention are derived from ethoxylated alcohols corresponding to general formula (I):
R¹O(CH₂CH₂O)ₙH (I)
in which R¹ and n are as defined above.

The alcohol mixtures are mixtures mainly of capric alcohol, lauryl alcohol, myristyl alcohol, cetyl alcohol and/or stearyl alcohol in the ratios by weight indicated. The mixtures can be obtained either by mixing the individual alcohols or by mixing corresponding alcohol mixtures. A preferred embodiment of the invention are citric acid ester mixtures of alcohols corresponding to formula (I), where R¹ is a linear alkyl group derived from a fatty alcohol mixture containing 65 to 75% by weight C₁₂, 20 to 30% by weight C₁₄, 0 to 5% by weight C₁₆ and 0 to 5% by weight C₁₈ alcohols. These alcohol mixtures on which the citric acid ester mixtures are based are commercially available alcohol mixtures, for example Dehydol LS™, a product of Cognis Deutschland GmbH & Co. KG. The fatty alcohol mixture has the following chain distribution in % by weight: C₁₀: 0-2%, C₁₂: 70-75%, C₁₄: 24-30%, C₁₆: 0-2%, and can be obtained, for example, from palm kernel oil or coconut oil.

Another preferred embodiment of the present invention are citric acid ester mixtures of ethoxylated alcohols corresponding to formula (I), where R¹ is a linear alkyl group derived from a fatty alcohol mixture containing 45 to 60% by weight C₁₂, 15 to 30% by weight C₁₄, 5 to 15% by weight C₁₆ and 8 to 20% by weight C₁₈ alcohol. These alcohol mixtures on which the citric acid ester mixtures are based are commercially available alcohol mixtures, for example Dehydol LT™, a product of Cognis Deutschland GmbH & Co. KG. The fatty alcohol mixture has the following chain distribution in % by weight: <C₁₂: 0-3%, C₁₂: 48-58%, C₁₄: 18-24%, C₁₆: 8-12%, C₁₈: 11-15%, >C₁₈: 0-1%, and can be obtained, for example, from palm kernel oil or coconut oil.

According to the invention, the degree of ethoxylation n is a number of 6 to 8 which may be an integer or a broken number.

Ethoxylation products of fatty alcohol mixtures containing 45 to 60% by weight C₁₂, 15 to 30% by weight C₁₄, 5 to 15% by weight C₁₆ and 8 to 20% by weight C₁₈ alcohol with 6 to 8 mol ethylene oxide and, more especially, the ethoxylation product of Dehydol LT™ with 7 mol ethylene oxide are particularly advantageous.

The (fatty) alcohol mixtures may contain small amounts of short-chain or relatively long-chain alcohols, preferably less than 10% by weight and, more particularly, 5% by weight in total, based on alcohol mixtures.

The citric acid ester mixtures according to the invention are mixtures of isomeric compounds corresponding to general formula (II): 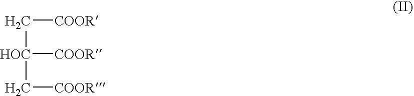
in which R′, R″, R′″ stand for X and/or an ethoxylated alkyl group R¹ with the meaning defined for formula (I), the distribution of the substituents R′, R″ and R′″ having to be such that the ratio by weight of monoester to diester is in the range from 3:1 to 10:1. In a preferred embodiment, the ratio by weight of monoester to diester is in the range from 5:1 to 8:1.

Accordingly, the citric acid ester mixtures according to the invention compulsorily contain mono- and diesters, preferably in quantities of 50 to 90% by weight and more particularly in quantities of 60 to 80% by weight, expressed as mono- and diesters and based on mixture. The mixtures may also contain triesters and free citric acid as the balance to 100% by weight. However, the mixtures preferably contain little free citric acid, preferably less than 10% by weight, based on mixtures.

Accordingly, the citric acid esters according to the invention are mainly partial esters of citric acid which still contain at least one free carboxyl group. The esters may therefore also be acidic esters or neutralization products thereof and X in formula (II) may be hydrogen or a cation. The partial esters are then preferably present in the form of alkali metal, alkaline earth metal, ammonium, alkylammonium, alkanolammonium and/or glucammonium salts (i.e. X=alkali metal, alkaline earth metal, ammonium, alkylammonium, alkanolammonium and/or glucammonium ion).

To produce the citric acid esters according to the invention, the citric acid must be esterified with the alcohol ethoxylates of formula (I) in a molar ratio of 0.9:1 to 1.1:1 and more particularly 1:1.

The process conditions as such correspond to the prior art. It can be essential to carry out the reaction in a nitrogen atmosphere. In addition, it can be of advantage to adjust a reaction temperature of 150 to 170° C. and preferably 160° C. The citric acid ester mixtures according to the invention are obtained as the end product. The esters may be present in free form or as salts. In general, a small percentage of the citric acid, preferably less than 10% by weight, remains unesterified for process-related reasons. Reaction products containing at most 8% and, more particularly, at most 5% unesterified citric acid are particularly preferred.

The acid value of the products obtained in accordance with the invention is preferably in the range from 120 to 180; the saponification value is in the range from 200 to 280 (all measurements to DIN).

The citric acid ester mixtures according to the invention can be formulated with other surfactants, advantageously with anionic and/or nonionic surfactants.

Surfactants

These other surfactants may be nonionic, anionic, cationic and/or amphoteric/zwitterionic surfactants. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, alkyl glucose carboxylates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds and esterquats, more particularly quaternized fatty acid trialkanolamine ester salts. Typical examples of amphoteric or zwitterionic surfactants are alkylbetaines, alkylamidobetaines, amino-propionates, aminoglycinates, imidazolinium betaines and sulfobetaines. Particularly preferred nonionic surfactants are inter alia the alkyl polyglycosides.

Particularly preferred nonionic surfactants are inter alia the alkyl polyglycosides. Particularly suitable anionic surfactants include the alkyl and/or alkenyl sulfates and the alkylether sulfates although the choice of nonionic and/or anionic surfactants is by no means limited to such surfactants.

Alkyl and/or alkenyl sulfates, which are often also referred to as fatty alcohol sulfates, are understood to be the sulfation products of primary alcohols which correspond to formula (III):
R²O—SO₃M (III)
in which R² is a linear or branched, aliphatic alkyl and/or alkenyl group containing 6 to 22 carbon atoms and preferably 12 to 18 carbon atoms and M is an alkali metal and/or alkaline earth metal, ammonium, alkyl ammonium, alkanolammonium or glucammonium. Typical examples of alkyl sulfates which may be used in accordance with the invention are the sulfation products of caproic alcohol, caprylic alcohol, capric alcohol, 2-ethyl hexyl alcohol, lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol and erucyl alcohol and the technical mixtures thereof obtained by high-pressure hydrogenation of technical methyl ester fractions or aldehydes from Roelen's oxo synthesis. The sulfation products may advantageously be used in the form of their alkali metal salts and particularly their sodium salts. Alkyl sulfates based on C_{16/18} tallow fatty alcohols or vegetable fatty alcohols of comparable C chain distribution in the form of their sodium salts are particularly preferred.

Alkyl ether sulfates (“ether sulfates”) are known anionic surfactants which, on an industrial scale, are produced by SO₃ or chlorosulfonic acid (CSA) sulfation of fatty alcohol or oxoalcohol polyglycol ethers and subsequent neutralization. Ether sulfates suitable for use in accordance with the invention correspond to formula (IV):
R³O—(CH₂CH₂O)ₘSO₃Z (IV)
in which R³ is a linear or branched alkyl and/or alkenyl group containing 6 to 22 carbon atoms, m is a number of 1 to 10 and Z is an alkali metal and/or alkaline earth metal, ammonium, alkylammonium, alkanolammonium or glucammonium. Typical examples are the sulfates of addition products of on average 1 to 10 and more particularly 1 to 5 mol ethylene oxide onto caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical mixtures thereof in the form of their sodium and/or magnesium salts. The ether sulfates may have both a conventional homolog distribution and a narrow homolog distribution. It is particularly preferred to use ether sulfates based on adducts of on average 2 to 3 mol ethylene oxide with technical C_{12/14} or C_{12/18} coconut fatty alcohol fractions in the form of their sodium and/or magnesium salts.

Alkyl and alkenyl oligoglycosides are known nonionic surfactants which correspond to formula (V):
R⁴O-[G]ₚ (V)
where R⁴ is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry. The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (IV) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational perspective. The alkyl or alkenyl group R⁴ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₋₂ alcohol as an impurity, and also alkyl oligoglucosides based on technical C_{9/11} oxoalcohols (DP=1 to 3) are preferred. In addition, the alkyl or alkenyl group R⁴ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} coconut oil fatty alcohol having a DP of 1 to 3 are preferred.
Commercial Applications

The citric acid ester mixtures according to the invention may be used on their own but, more particularly, are used in admixture with one or more of the above-mentioned surfactants for the production of foaming, skin-friendly cosmetic preparations.

The cosmetic preparations may be water-free or water-containing formulations. More particularly, the compounds are used in hair shampoos, hair lotions, foam baths, shower baths, creams, gels, lotions, alcoholic and aqueous/alcoholic solutions, emulsions, wax/fat compounds, stick preparations, powders or ointments. The citric acid ester mixtures according to the invention may also be used in combination with other auxiliaries and additives typically encountered in cosmetic products, such as, for example, oil components, emulsifiers, superfatting agents, pearlizing waxes, consistency factors, thickeners, polymers, silicone compounds, fats, waxes, lecithins, phospholipids, stabilizers, biogenic agents, deodorizers, antiperspirants, antidandruff agents, film formers, swelling agents, UV protection factors and the like.

The citric acid ester mixtures are preferably used in quantities of 0.1 to 20% by weight and more particularly in quantities of 0.5 to 10% by weight, based on cosmetic preparation.

For the cosmetic preparations, mixtures of APG compounds corresponding to formula (V) and the citric acid ester mixtures according to the invention, in which the ratio by weight of the APGs to the citric acid ester mixtures is in the range from 3:1 to 1:3, show particularly advantageous properties.

Water-containing formulations are particularly preferrred, particularly if they are mildly acidic, preferably with a pH of 5 to 6.5.

### EXAMPLES

Substances used:

1. Dehydol LT 7™, a product of Cognis Deutschland GmbH & Co. KG, is a fatty alcohol mixture ethoxylated with 7 mol ethylene oxide. The fatty alcohol mixture has the following chain distribution in % by weight: <C12:0-3%; C12: 48-58%; C14: 18-24%; C16: 8-12%; C18: 11-15%; >C18: 0-1%.

2. Dehydol LS 6™, a product of Cognis Deutschland GmbH & Co. KG, is a fatty alcohol mixture ethoxylated with 6 mol ethylene oxide. The fatty alcohol mixture has the following chain distribution in % by weight: C10:0-2%; C12: 70-75%; C14: 24-30%; C16: 0-2%.

3. A fatty alcohol mixture ethoxylated with 10 mol ethylene oxide. The fatty alcohol mixture has the following chain distribution in % by weight: <C12:0-3%; C12: 48-58%; C14: 18-24%; C16: 8-12%; C18: 11-15%; >C18: 0-1%.

4. Dehydol 04™, a product of Cognis Deutschland GmbH & Co. KG, is an octanol ethoxylated with 4 mol ethylene oxide.

### Example 1

### Citric acid ester of a C₁₂₋₁₈ alcohol+7EO; monoester:diester 6:1

In a stirred reactor, 28.05 kg (0.146 mol) water-free citric acid and 75.16 kg (0.146 Kmol) Dehydol LT 7™ were heated under nitrogen to 160° C. and stirred at that temperature until the theoretical quantity of water had been released (5.5 hours). A light yellow, clear and liquid product with the following characteristics was obtained:

Characteristics of the Citric Acid Ester of Example 1

| | |
|---|---|
| saponification value | 222 |
| acid value | 132 |
| free citric acid | 2.8% by weight |
| ratio by weight of mono- to diester | 6:1 |

### Example 2

### Citric acid ester of a C_{12/14} alcohol+6EO; monoester:diester 6:1

As in Example 1, 249.7 g (1.3 mol) water-free citric acid and 607.9 g (1.3 mol) Dehydol LS 6™ were heated under nitrogen to 160° C. in a stirred reactor and stirred at that temperature until the theoretical quantity of water had been released (2 hours). A light yellow, clear and liquid product with the following characteristics was obtained:

Characteristics of the Citric Acid Ester of Example 2

| | |
|---|---|
| saponification value | 253 |
| acid value | 173 |
| free citric acid | 7.1% by weight |
| ratio by weight of mono- to diester | 6:1 |

### Comparison Example 1

### Citric acid ester of a C₁₂₋₁₈ alcohol+7EO; monoester:diester 1:1

In a stirred reactor, 172.9 g (0.9 mol) water-free citric acid and 905.8 g (1.8 mol) Dehydol LT 7™ were heated under nitrogen to 160° C. and stirred at that temperature until the theoretical quantity of water had been released (7 hours). A yellow, bright and liquid product with the following characteristics was obtained:

Characteristics of the Citric Acid Ester of Comparison Example 1

| | |
|---|---|
| saponification value | 126.1 |
| acid value | 48.6 |
| free citric acid | 0.2% by weight |
| ratio by weight of mono- to diester | 1:1 |

### Comparison Example 2

### Citric acid ester of a C₁₂₋₁₈ alcohol+10EO; monoester:diester 6:1

As in Example 1, 0.9 mol water-free citric acid and 0.9 mol of the fatty acid mixture ethoxylated with 10 mol ethylene oxide (3^{rd} of the substances used) were heated under nitrogen to 160° C. in a stirred reactor and stirred at that temperature until the theoretical quantity of water had been released (2.5 hours). A light yellow, clear and liquid product with the following characteristics was obtained:

Characteristics of the Citric Acid Ester of Comparison Example 2

| | |
|---|---|
| saponification value | 214.6 |
| acid value | 139.7 |
| free citric acid | 6.1% by weight |
| ratio by weight of mono- to diester | 6:1 |

### Comparison Example 3

### Citric acid ester of a C₈ alcohol+4EO; monoester:diester 6:1

As in Example 1, 0.9 mol water-free citric acid and 0.9 mol Dehydol 04™ were heated under nitrogen to 160° C. in a stirred reactor and stirred at that temperature until the theoretical quantity of water had been released (2 hours). A light yellow, clear and liquid product with the following characteristics was obtained:

Characteristics of the Citric Acid Ester of Comparison Example 3

| | |
|---|---|
| saponification value | 369.0 |
| acid value | 230 |
| free citric acid | 9.6% by weight |
| ratio by weight of mono- to diester | 6:1 |

The saponification value (SV) was determined to DGF C-V3.

The acid value (AV) was determined to DIN 53402.

### Performance Tests

Determination of Foam Behavior

To determine foaming behavior, the foaming kinetics after 30 seconds and the foam potential after 60, 90, 120, 150 s and 180 s were measured by the rotor foam method (DIN 13996 in preparation). The rotor foam tester consists of a heatable, double-walled cylindrical glass vessel with an internal diameter of 17.5 cm. A scale in mm is provided on the cylindrical glass vessel for reading off the foam height and the liquid level. In addition, the glass vessel is provided with a Styropor lid which is used both to cover and to insulate the vessel. The stirrer consists of a special stirring head with a stirrer shaft 28 cm in length and 1 cm in diameter and a JK stirrer with a digital revolution counter. A thermostat, a stopwatch and a thermometer (digital) are also required.

The test solution was prepared with water of a certain hardness (150 dH).

200 ml of the sample preheated to 40±1° C. (0.5 g testsubstance/l; pH=6) were slowly poured in at the rim of the glass vessel which was covered with the Styropor lid when the required temperature of 40±1° C. had been reached. The rotor speed was 1300 r.p.m.

The first foam height value was determined after 30 seconds. To this end, the stirrer was switched off for at most 10 seconds. The foam volume was then determined after 60 and 180 seconds.

TABLE 1
Foam behavior of citric acid esters

| | | |
|---|---|---|
| Substance | Concentration | Foaming behavior |

| | | | |
|---|---|---|---|
| Citric acid ester of a | 0.5 g/l | 30 s | 211 ml |
| C₁₂₋₁₈ alcohol + 7EO; | | 60 s | 339 ml |
| monoester:diester | | 90 s | 477 ml |
| 6:1/Example 1 | | 120 s | 606 ml |
| | | 150 s | 781 ml |
| | | 180 s | 787 ml |
| Citric acid ester of a | 0.5 g/l | 30 s | 265 ml |
| C_{12/14} alcohol + 6EO; | | 60 s | 479 ml |
| monoester:diester | | 90 s | 703 ml |
| 6:1/Example 2 | | 120 s | 796 ml |
| | | 150 s | 811 ml |
| | | 180 s | 813 ml |
| Citric acid ester of a | 0.5 g/l | 30 s | 150 ml |
| C₁₂₋₁₈ alcohol + 7EO; | | 60 s | 204 ml |
| monoester:diester 1:1/ | | 90 s | 275 ml |
| Comparison Example 1 | | 120 s | 328 ml |
| | | 150 s | 364 ml |
| | | 180 s | 398 ml |
| Citric acid ester of a | 0.5 g/l | 30 s | 202 ml |
| C₁₂₋₁₈ alcohol + 10EO; | | 60 s | 291 ml |
| monoester:diester 6:1/ | | 90 s | 383 ml |
| Comparison Example 2 | | 120 s | 455 ml |
| | | 150 s | 489 ml |
| | | 180 s | 532 ml |
| Citric acid ester of a C₈ | 0.5 g/l | 30 s | 163 ml |
| alcohol + 4EO; | | 60 s | 224 ml |
| monoester:diester 6:1/ | | 90 s | 296 ml |
| Comparison Example 3 | | 120 s | 354 ml |
| | | 150 s | 382 ml |
| | | 180 s | 405 ml |

It is clear from Table 1 that the citric acid esters according to the invention with the selected monoester:diester contents show distinctly better foam behavior than citric acid esters with higher diester contents (Example 1 against Comparison Example 1). In addition, the citric acid esters according to the invention with the selected degrees of ethoxylation show better foam behavior than those with higher degrees of ethoxylation (Example 1 against Comparison Example 2) or even with shorter alcohol chains (Examples 1 and 2 against Comparison Example 3) both in regard to foaming kinetics and after relatively long times.

Determination of Irritation Potential by the RBC Test

The RBC Test was carried out by W. Pape and U. Hoppe's method (Arzneim.-Forsch./Drug Res. 40(1), No. 4 (1990); pp. 498 et seq).

TABLE 2
RBC Test

| Example | Compound | L/D | Classification |
|---|---|---|---|
| 1 | Citric acid ester of a C₁₂₋₁₈ alcohol + 7EO; | >100 | Non-lachrimatory |
| | monoester:diester 6:1 | | |
| 2 | Citric acid ester of a C_{12/14} alcohol + 6EO; | >100 | Non-lachrimatory |
| | monoester:diester 6:1 | | |
| Comparison | Citric acid ester of a C₁₂₋₁₈ alcohol + 7EO; | 4.9 | Moderately |
| Example 1 | monoester:diester 1:1 | | lachrimatory |
| Comparison | Citric acid ester of a C₁₂₋₁₈ alcohol + 10EO; | >100 | Non-lachrimatory |
| Example 2 | monoester:diester 6:1 | | |
| Comparison | Citric acid ester of a C₈ alcohol + 4EO; | 6.4 | Moderately |
| Example 3 | monoester:diester 6:1 | | lachrimatory |

It is clear from Table 2 that the citric acid esters according to the invention are non-lachrimatory and, hence, more compatible than comparable citric acid esters with higher diester contents (Comparison Example 1) or with shorter alkyl chains (Comparison Example 3).

1-11. (canceled)

12. A mixture of citric acid esters of ethoxylated alcohols of the formula:
R¹O(CH₂CH₂O)ₙH (I)
in which R¹ is an alkyl group and n is a degree of ethoxylation,
wherein, R¹ is a linear alkyl group of a residue of a fatty alcohol mixture containing 45 to 75% by weight C₁₂, 15 to 35% by weight C₁₄, 0 to 15% by weight C₁₆ and 0 to 20% by weight C₁₈ alcohol and n is a number of 5 to 9, with the proviso that a ratio by weight of monoester to diester in the mixture of citric acid esters is in a range from 3:1 to 10:1.

13. The mixture of citric acid esters of claim 12, wherein, the ratio by weight of monoester to diester is in the range from 5:1 to 8:1.

14. The mixture of citric acid esters of claim 12, wherein, n is a number of 6 to 8.

15. The mixture of citric acid esters of claim 12, wherein, R¹ is the linear alkyl group of the residues of a fatty alcohol mixture containing 65 to 75% by weight C₁₂, 20 to 30% by weight C₁₄, 0 to 5% by weight C₁₆ and 0 to 5% by weight C₁₈ alcohol.

16. The mixture of citric acid esters of claim 12, wherein, R¹ is a linear alkyl group of the residue of a fatty alcohol mixture containing 45 to 60% by weight C₁₂, 15 to 30% by weight C₁₄, 5 to 15% by weight C₁₆ and 8 to 20% by weight C₁₈ alcohol.

17. The mixture of citric acid esters of claim 12, wherein, in formula (I), n is a number of 6 to 8 and R¹ is a linear alkyl group of the residue of a fatty alcohol mixture containing 45 to 60% by weight C₁₂, 15 to 30% by weight C₁₄, 5 to 15% by weight C₁₆ and 8 to 20% by weight C₁₈ alcohol.

18. A process for the production of a mixture of citric acid esters of ethoxylated alcohols of a formula:
R¹O(CH₂CH₂O)ₙH (I)
in which R¹ is a linear alkyl group of a residue of a fatty alcohol mixture containing 45 to 75% by weight C₁₂, 15 to 35% by weight C₁₄, 0 to 15% by weight C₁₆ and 0 to 20% by weight C₁₈ alcohol and n is a number of 5 to 9, with the proviso that a ratio by weight of monoester to diester in the mixture of citric acid esters is in a range from 3:1 to 10:1, wherein, the citric acid is esterified with the alcohol ethoxylates of formula (I) in a molar ratio of 0.9:1 to 1.1:1.

19. The process of claim 18 wherein the molar ratio is 1:1.

20. A foaming skin friendly cosmetic preparation comprising a mixture of citric acid esters of claim 12, optionally in admixture with other surfactants.

21. The cosmetic preparation of claim 20, further comprising at least one anionic surfactant selected from the group consisting of alkyl sulfates, alkenyl sulfates and alkylether sulfates.

22. The cosmetic preparation of claim 20, further comprising an alkyl polyglucoside nonionic surfactant.

23. The cosmetic preparation of claim 20, comprising from 0.1% to 20% by weight of the mixture of citric acid esters based on the weight of the cosmetic preparation.

24. The cosmetic preparation of claim 23 comprising from 0.5% to 10% by weight of the mixture of citric acid esters based on the weight of the cosmetic preparation.

25. The mixture of citric acid esters of claim 12 wherein the fatty alcohol mixture contains less than 10% by weight of fatty alcohols with a chain length greater than C₁₈ and fatty alcohols with a chain length less than C₁₂ based on the total amount of alcohols with a chain length greater than C₁₈ and less than C₁₂ based on the total weight of the fatty alcohol mixture.

26. The mixture of citric acid esters of claim 12 containing less than 10% by weight of unesterified citric acid.

27. The mixture of citric acid esters of claim 26 containing less than 5% by weight of unesterified citric acid.

28. The mixture of citric acid esters of claim 12 having an acid value of from 120 to 180.
